(19) United States
(12) Patent Application Publication
(10) Pub. No.: US 2024/0132817 A1
(43) Pub. Date: Apr. 25, 2024

(54) ACTUATOR HOLDER APPARATUS

(71) Applicant: Chiron On-Chip Biotechnologies B.V., Maastricht (NL)

(72) Inventor: Carlo Paggi, Maastricht (NL)

(73) Assignee: Chiron On-Chip Biotechnologies B.V., Maastricht (NL)

(21) Appl. No.: 18/490,846

(22) Filed: Oct. 19, 2023

Related U.S. Application Data
(60) Provisional application No. 63/417,694, filed on Oct. 20, 2022.

(51) Int. Cl. C12M 3/00; C12M 1/00; C12M 1/04; C12M 1/12

(52) U.S. Cl. CPC C12M 21/08; C12M 23/24; C12M 23/26; C12M 25/14

(57) ABSTRACT

The present apparatus is a hybrid system that can use human tissue and/or cells coupled with a hydrogel and can use tissue explants (biopsies). The system is implemented in an embodiment by use of an actuator and a holder. The actuator is implemented with a one or more of chambers of a plurality of shapes, with each chamber addressable by an associated single inlet. In one embodiment, the holder comprises one or more independent chambers where cells or biopsies can be placed. In one embodiment, the holder is coupled to the actuator via the use of guides, threads, and the like that allow one part to slide into the other.

This patent application claims priority to U.S. Patent Application 63/417,694 filed on Oct. 20, 2022, which is incorporated by reference herein in its entirety.

### BACKGROUND OF THE SYSTEM

Currently, the standard models to study disease and investigating possible solutions use in vitro and in vivo models. In vitro models utilize a simple 2D cell culture system or 3D spheroids. These models have many disadvantages when seeking to replicate or investigate tissue and structure (e.g., cartilage). In vitro models are poor at replicating the physiology and structure of the tissues and the organs within our body.

In vivo models use animals as a testing system. The most common animals are mice, rabbits, horses, pigs and monkeys. Although in vivo models are closer in the replication of a disease model such as arthritis, the behaviour of an in vivo model is still very different from the human physiology. Mice do not apply the same mechanical stimuli onto the joints when walking, which poorly translates in how the human cells experience stimuli. Additionally, the recovery of small animals from injury is faster compared to the human and this could lead to potential overestimation of the effect of an investigated treatment.

In the last two decades, a new in vitro solution has been developed, organ-on-chips. Organ-on-chips (OOCs) are miniaturized models which are able to replicate key characteristics of the human body, such as mechanical loading and biochemical stimuli, while using human cells, tissue, and other materials. These models are able to replicate the physiology and pathophysiology of a tissue or organ and are becoming the standard for drug development studies (nature.com/articles/s41573-020-0079-3). These advanced in vitro models allow a researcher to have real-time visualization and can be coupled with a sensing system to perform real time screening. Hence, OOCs offer a good alternative by increasing the complexity of the in vitro system while keeping the advantages of a humanized model.

Current approaches to OOC models to mimic mechanical stimulation include use of hydrogel/membrane constructs, or well plate constructs. These approaches have the disadvantage of difficulty in accessing the construct, complex handling requiring highly trained personnel, lack of use of biopsies, no current application of compressive forces on biopsies, and the like.

### SUMMARY

The present apparatus is a hybrid system that can use human tissue and/or cells coupled with a hydrogel and can use tissue explants (biopsies). The system is implemented in an embodiment by use of an actuator and a holder. The actuator is implemented with a one or more of chambers of a plurality of shapes, with each chamber addressable by an associated single inlet. In one embodiment, the holder comprises one or more independent chambers where cells or biopsies can be placed. In one embodiment, the holder is coupled to the actuator via the use of guides, threads, and the like that allow one part to slide into the other.

### BRIEF DESCRIPTION OF THE DRAWINGS

FIG. 1 is an embodiment of an actuator with a single inlet for each chamber.

FIG. 2 is an embodiment of an actuator with a single inlet for all chambers.

FIG. 3 illustrates possible shapes of the actuator chambers.

FIG. 4 illustrates movement of a flexible membrane of a chamber in an embodiment,

FIG. 5 illustrates a top view of a holder in an embodiment.

FIG. 6 illustrates a bottom view of the holder of FIG. 5.

FIG. 7 illustrates a side view of a chamber of a holder.

FIG. 8 illustrates the side view of FIG. 7 with medium and nutrients being added.

FIG. 9 illustrates a perspective view of the actuator showing the locking rails.

FIG. 10 illustrates a perspective view of the holder sowing the locking slots.

FIG. 11 illustrates a cross sectional view of an actuator in an embodiment.

FIG. 12 illustrates a cross sectional view of an actuator chamber of FIG. 11 in an embodiment.

FIG. 13 illustrates a top view of a well plate.

FIG. 14 illustrates a perspective view of a holder and actuator assembly for insertion into a well of a well plate.

FIG. 15 illustrates a view of the actuator of FIG. 14.

FIG. 16 illustrates a view of the holder of FIG. 14.

### DETAILED DESCRIPTION OF THE SYSTEM

The apparatus is comprised of an actuator and a holder. The actuator is comprised of one or more chambers attached to one or more inlets. The chambers can be filled with fluid (liquid or gas) via the inlet to cause expansion of the chamber, and thereby apply compression to a corresponding biological sample in a corresponding holder.

Actuator

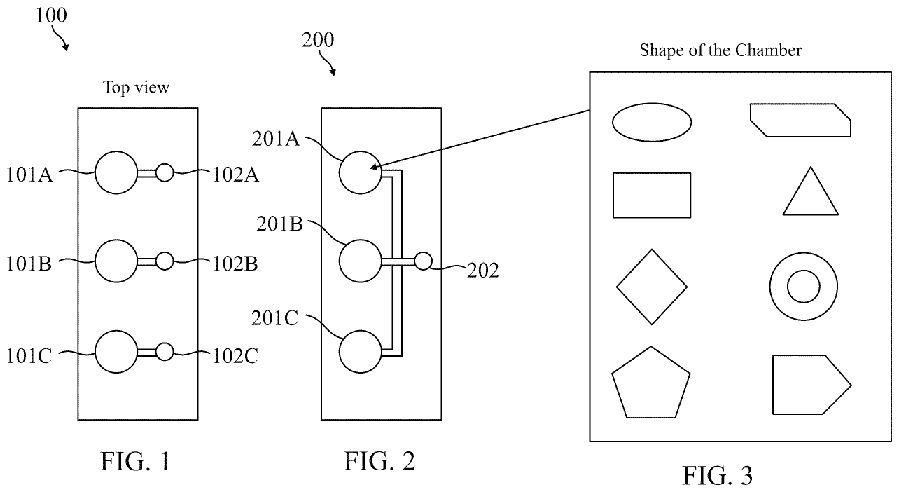

FIG. 1 illustrates an embodiment of an actuator where each chamber has its own corresponding inlet. The actuator chambers may be the same size or different size based on the intended use, biological samples used, and the like. This allows each chamber to operate independently, with different compressive force applied by each chamber, providing unique data gathering opportunities for a researcher. The actuator 100 includes chambers 101A, 101B, and 101C. Each chamber has its own corresponding fluid inlet 102A, 102B, and 102C respectively. The fluid may be a liquid or gas or any other suitable substance that can be introduced in the chamber to cause expansion and provide force on a sample.

FIG. 2 illustrates an embodiment of an actuator with a shared inlet. The actuator 200 includes chambers 201A, 201B, and 201C. A single inlet 202 is coupled to all three chambers. In this embodiment, the force applied by the chambers is the same for all three chambers, allowing comparison of different tissue samples and/or treatments under the same conditions.

FIG. 3 illustrates example chamber cross sectional shapes. Each shape can be used for a different effect, and different shapes can be used on the same actuator as desired. It should be noted that the use of three chambers is for example only, and any number of chambers can be utilized without departing from the scope and spirit of the present system.

In one embodiment, the actuator is made of flexible material (e.g., polydimethylsiloxane). It can be fabricated using soft-lithography, photo-lithography, hot embossing, injection molding, or any other process that is able to work with plastic materials. The actuator is transparent in one embodiment to allow real-time visualization but other colors and opacity can be adopted as desired.

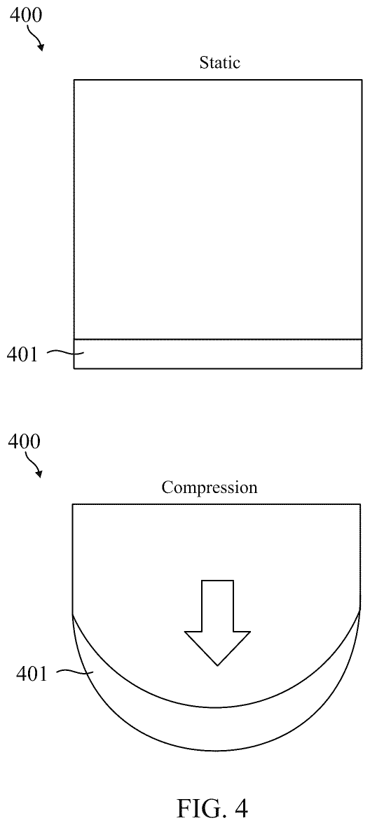

FIG. 4 illustrates a side sectioned view of an actuator chamber 400 in an embodiment. In the top view, the chamber 400 is in a static state, and a flexible membrane 401 on the bottom of the chamber is straight and applying no force to a sample located below the membrane. In the bottom of FIG. 4, a fluid is applied to the actuator chamber via an inlet, causing the flexible membrane 401 to deform into a convex shape, applying compressive force on any sample located below the actuator chamber. The greater the pressure from the fluid, the greater the deformation of the membrane.

Holder

The holder consists of a plastic material (polystyrene, PET, transparent resin, etc.) in one embodiment. It can be fabricated using soft-lithography, photo-lithography, hot embossing, injection molding or any other process that is able to work with plastic materials. The holder is transparent to allow real-time visualization in one embodiment, but other colors can be adopted.

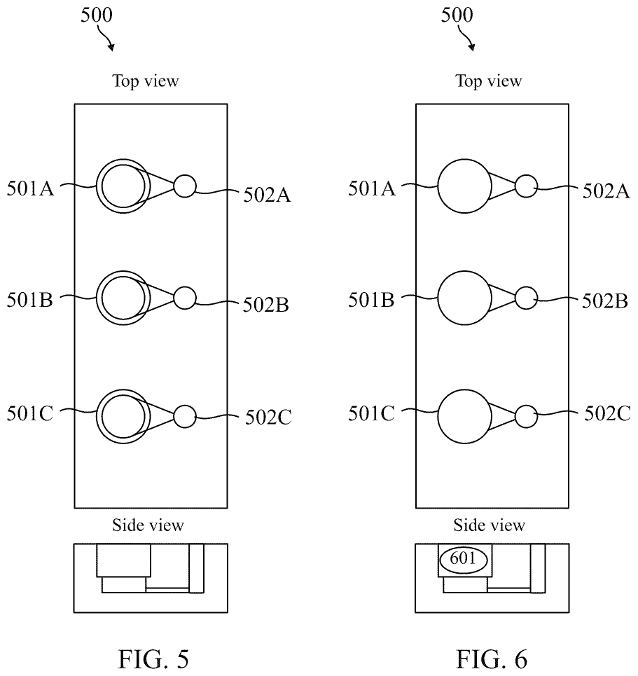

In one embodiment, as illustrated in FIG. 5 and FIG. 6, the holder 500 consist of 3 independent culture chambers 501A, 501B, and 501C where both hydrogels containing cells or biopsies (or any other type of cellular configurations) can be placed. IT should be noted that a holder may have one or more culture chambers without departing from the scope and spirit of the system. The sample can be human, animal, or plant based as desired. It may be cell aggregates, organoids, and the like.

From a second inlet 502A, 502B, or 502C, it is possible to access to the hydrogels or biopsies and provide nutrients or other biochemical compounds (e.g. drugs, cytokines). The culture chamber can vary in size from 4-5 mm in diameters to 10-15 mm depending on the sample used. The height of the same chambers can vary between 2 to 200 micrometres again depending on the size of the sample used. The second inlet can vary in size depending on the type of pipette use for injecting the nutrients. Standard sizes could vary between 0.2 to 2 mm. FIG. 6 illustrates the system with a sample 601 (biopsy or hydrogel plus cells) in the chamber.

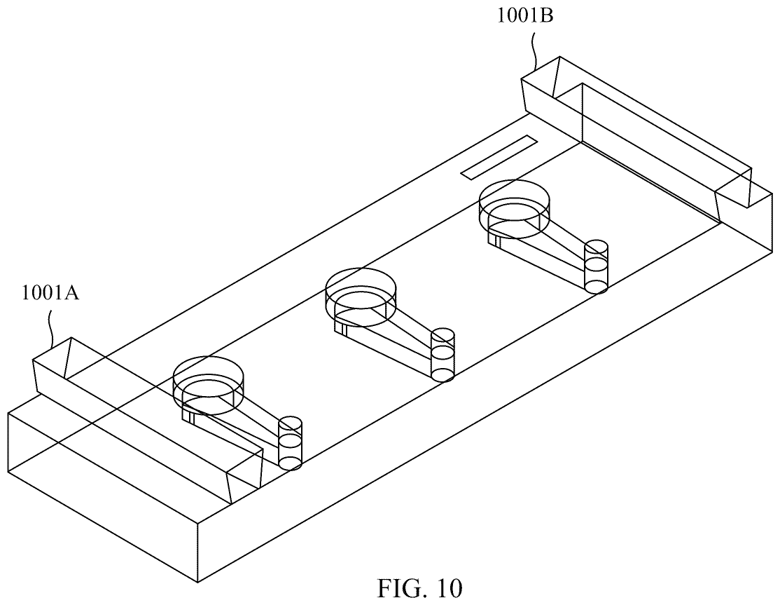

The Actuator and Holder can be coupled to each other in one embodiment using guides or threaded structures, and the like, that register the two pieces with each other. FIG. 9 illustrates the Actuator and shows recessed female guides 901A and 901B. The guides in one embodiment are shaped to be more narrow at one edge and wider at the other edge. The Holder in FIG. 10 includes corresponding male guides 1001A and 1001B that register with the female guides of the Actuator to join the two pieces together securely and to ensure proper registration of the sample chambers. The guides may be asymmetrical so to prevent the Actuator from being inserted in the wrong direction relative to the Holder. The deforming chamber of the Actuator should align with the sample chamber of the Holder. The cross sectional shape of the guides can vary and be of any suitable male and female shape that allows joining of the two components.

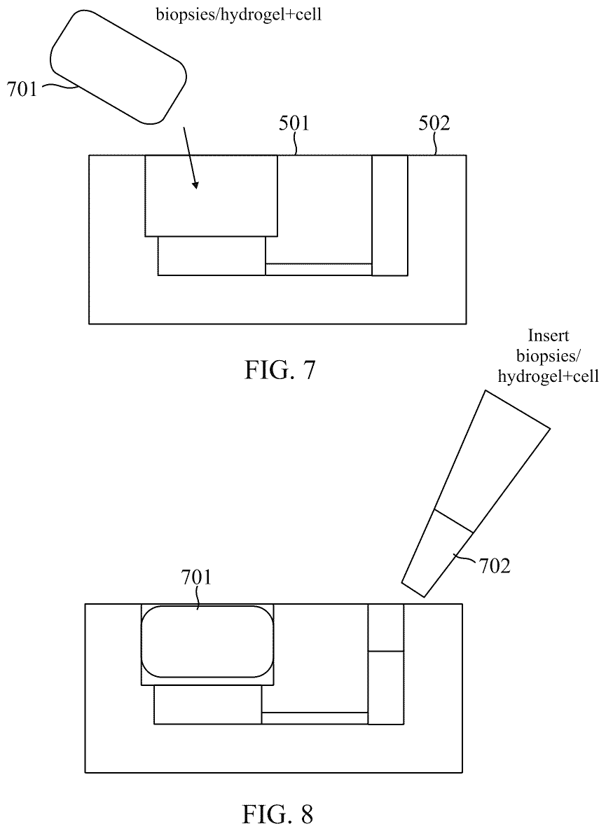

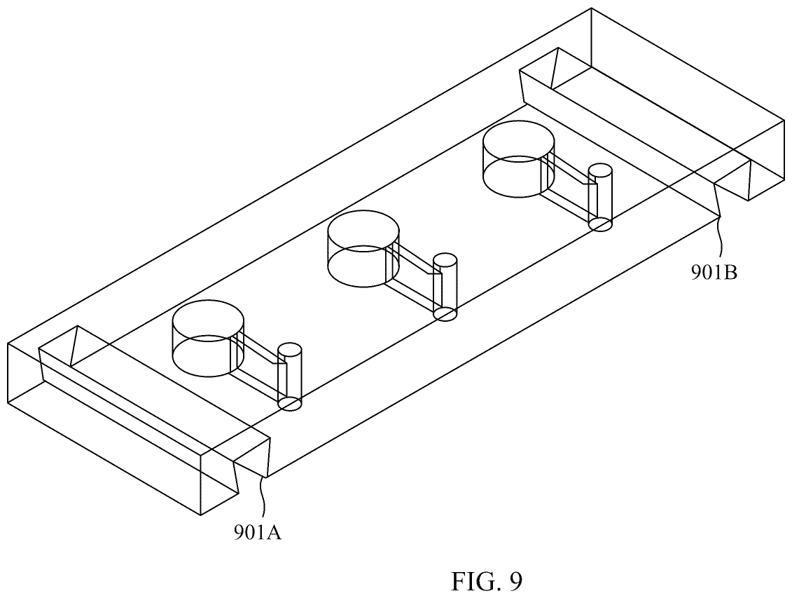

FIG. 7 illustrates a side view of the holder with a biopsy 701 being inserted into a chamber 501. As shown in FIG. 8, medium and/or nutrients can be added to the biopsy via a pipette 701 into inlet 502.

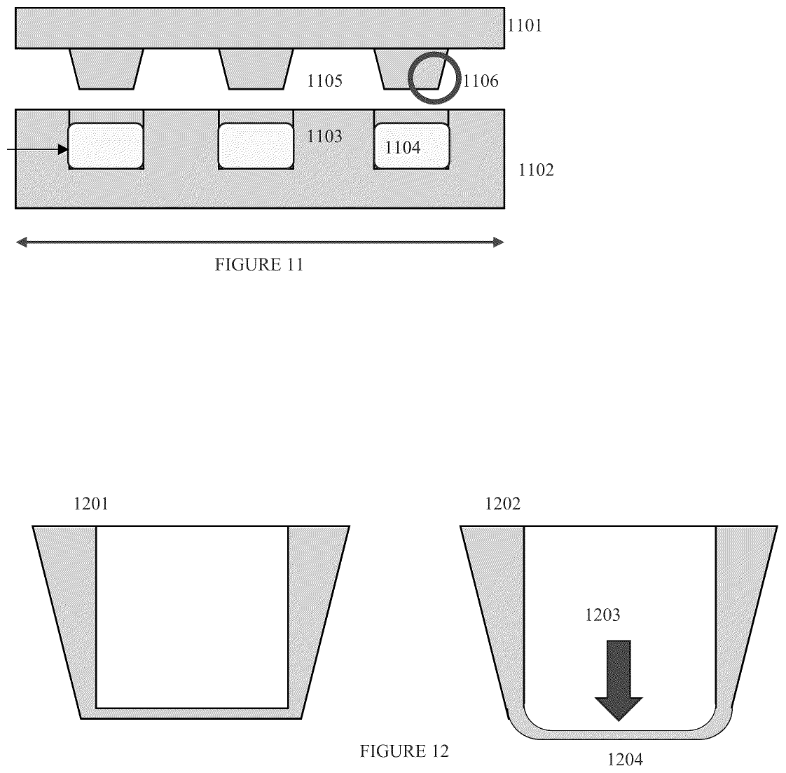

FIG. 11 illustrates a side view of an actuator and holder in an embodiment. The actuator comprises a plurality of actuator chambers, such as actuator chamber 1105. The holder 1102 comprises a plurality of sample chambers such as sample chamber 1103. A biopsy sample 1104 can be placed in one or more of the sample chambers 1103. The actuator chamber 1105 includes a beveled or slanted side 1106. The actuator chamber in one embodiment is shaped as a truncated cone with a narrow end engaging the sample chamber 1103. The slanted shape allows the actuator chamber 1106 to fit deeply into the sample chamber to ensure engagement with the sample, so that force can be reliable applied to the sample. The depth and width of the truncable cone can vary as the application requires. In one embodiment, the sample depth is selected so that is only touching the bottom face of the actuator and then mechanical actuation is applied upon application of pressure in the chamber of the actuator.

FIG. 12 illustrates a cross sectional view of the actuator chamber of FIG. 11 with and without force applied. Actuator chamber 1201 is shown with no force applied. The chamber is hollow inside, allowing fluid or gas to be introduced to the interior. Actuator chamber 1202 illustrates the use of force 1203 applied by introducing a fluid or gas into the actuator chamber. The bottom 1204 of the chamber 1202 is extended in the direction of the force, which in turn will apply force to the sample in the sample chamber. In one embodiment, the system may use a mechanical actuator to provide force to the actuator chamber, instead of using fluid or gas.

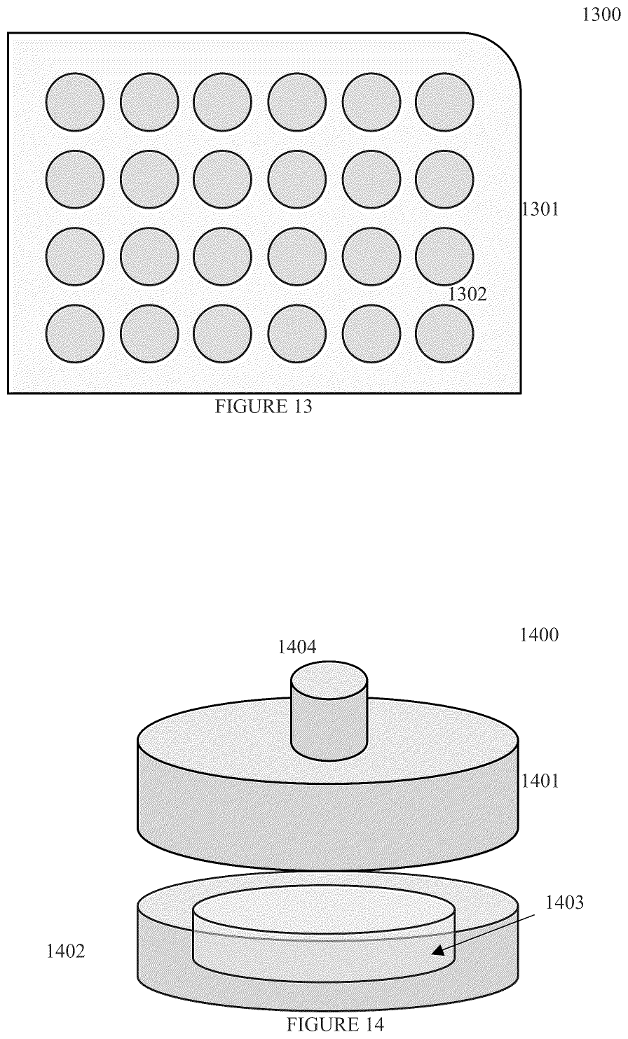

An embodiment of the system for use with standard well plates is illustrated in FIG. 13 and FIG. 14. Referring first to FIG. 13, a top view of a well plate is illustrated. The well plate 1300 is a tray 1301 having a plurality of cylindrical wells 1302 formed thereon. Typically, the wells receive solid or fluid samples for assays or other functions. The number of wells can vary as desired (e.g., 6 to 96 wells) but any number may be utilized.

FIG. 14 is a perspective view of a sample holder and actuator assembly 1400 in an embodiment of the system. The assembly 1400 comprises an actuator 1401 and sample holder 1402. The sample holder is round in shape with a hollow center region that can receive a sample plug 1403. The hollow region may be circular in an embodiment, but may have other cross sectional shapes as desired.

The sample holder 1402 and actuator 1401 are circular in cross section and of a size to fit into a well of a well plate. The actuator 1401 includes a member 1404 for introducing force to the actuator, such as fluid or gas. The actuator 1401 has a hollow portion inside and has a bottom layer that is flexible and can deform in the presence of increased pressure, to thereby apply force to the sample plug 1403 in the sample holder 1402.

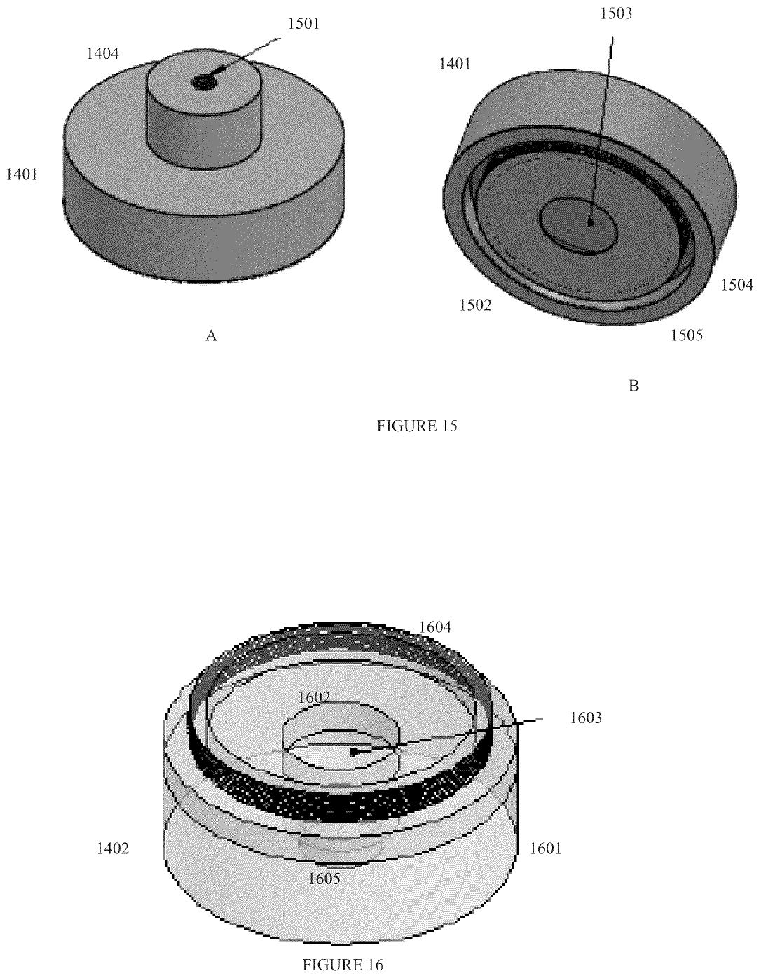

In one embodiment, the actuator 1401 engages the sample holder 1402 by threaded members that engage to join the two components together. The components could also use guides, pressure fit, screws, clips, tabs, and the like to join the two components together. FIG. 15 illustrates a view of the actuator of FIG. 14. Side A shows a top perspective view of Actuator 1401 with the member 1404. An opening 1501 in member 1404 is used to introduce fluid or gas to the interior of actuator 1401 during operation to apply force to the sample plug.

Side B of FIG. 15 shows a bottom perspective view of actuator 1401. The actuator 1401 includes a bottom surface 1502 that includes a flexible membrane 1503 that can deform in response to fluid, gas, or mechanical force applied through opening 1501. There is a threaded gap 1504 between the bottom surface 1502 and an outer wall 1504 of the actuator 1401. This threaded gap 1504 will engage with the sample holder 1402 to join them together in a sealed relationship.

FIG. 16 illustrates a view of the holder 1402 of FIG. 14. The sample holder 1402 has a cylindrical body 1601 and hollow sample chamber 1603 to receive the sample plug through upper surface 1602. A threaded ring 1604 is formed on the upper surface 1602, having a diameter smaller than the diameter of the body 1601. The threaded ring engages the threaded gap 1504 of actuator 1401 to join the two pieces together securely, for insertion into a well 1302 of well tray 1300. An opening 1605 in a bottom surface of sample holder 1402 allows nutrients, drugs, or other substances to be introduced to the sample plug as desired.

The system can be implemented with different shapes of each chamber, and can allow the use of different biopsies in each chamber, providing additional complexity while maintaining ease of use. In one embodiment, the system can implement sensors or accessories. For example, an O-ring can be added to reduce the diameter of the chamber to allow for smaller samples to be held in place.

What is claimed is:

1. An apparatus comprising:
i. a first component having a sample holder formed therein for holding a biological sample;
ii. a second component having an actuator formed therein for applying force to the biological sample when activated and removing the force when not activated;
iii. engaging means for coupling the first component with the second component to register the actuator with the sample holder.

2. The apparatus of claim 1 further including an inlet for introducing therapies to the biological sample.

3. The apparatus of claim 1 wherein the actuator has a hollow portion having flexible membrane such that an introduction of fluid or gas to the hollow portion causes deflection of the membrane towards the biological sample, applying force to the sample.

4. The apparatus of claim 1 wherein the engaging means comprises guides on the first component and second component formed so that the two components can only be engaged in one manner.

5. The apparatus of claim 4 wherein the engaging means comprises a rail and a slot.

6. The apparatus of claim r wherein the engaging means comprises a male threaded member and a female threaded member.

7. The apparatus of claim 1 wherein the apparatus can be placed in a well of a well plate.

8. The apparatus of claim 1 wherein a cross section of the sample holder is circular.

9. The apparatus of claim wherein the actuator is in the form of a truncated cone.

10. The apparatus of claim 1 further including a plurality of sample holders on the first component and a corresponding number of actuators on the second component.
